(19) United States
(12) Patent Application Publication
(10) Pub. No.: US 2020/0187435 A1
(43) Pub. Date: Jun. 18, 2020

(54) PROCEDURE FOR INSTALLING AN ELECTRONIC SENSOR IN A PLANT

(71) Applicant: FloraPulse C, Davis, CA (US)

(72) Inventors: Michael Santiago, Davis, CA (US); Justin K. Fontes, Woodland, CA (US); Kenneth A. Shackel, Davis, CA (US)

(21) Appl. No.: 16/712,934

(22) Filed: Dec. 12, 2019

Related U.S. Application Data
(60) Provisional application No. 62/778,690, filed on Dec. 12, 2018.

(51) Int. Cl. A01G 25/16; G01N 33/00

(52) U.S. Cl. CPC A01G 25/167; G01N 33/0098

(57) ABSTRACT

Methods for installing a plant hydration status sensor into a plant to provide reliable plant hydration data are disclosed. One contemplated method comprises creating a hole from an exterior surface of the plant to a depth that exposes the water tissue. Once the hole is created, a slurry can be applied, and a sleeve and a plant hydration status sensor can be inserted into the hole. A sealant can be applied about the plant hydration status sensor and the sleeve to prevent or reduce sensor interference due to outside environment conditions.

This application claims priority to U.S. provisional application: Ser. No. 62/778,690 filed Dec. 12, 2018, entitled “Procedure for Installing an Electronic Chip in a Plant.” All extrinsic materials identified herein are incorporated by reference in their entirety.

### FIELD OF THE INVENTION

The field of the invention is plant hydration monitoring.

### BACKGROUND

The background description includes information that can be useful in understanding the present invention. It is not an admission that any of the information provided herein is prior art or relevant to the presently claimed invention, or that any publication specifically or implicitly referenced is prior art.

Over- or under-irrigation of crops can result in loss in quality or yield. Several systems and methods for determining plant hydration have been developed. For example, some technologies estimate plant hydration using soil data. WO9804915 (Nomura), for example, teaches a soil probe disposed in a single point in the soil near a plant. However, such method of measuring plant hydration might or might not accurately reflect actual plant hydration because the probe is inserted into the soil instead of directly into the plant. Even if multiple soil probes are utilized, the method provides only soil data, not plant data.

Other technologies do directly measure plant hydration, but are susceptible to inaccuracies due to expulsion of the probe via plant exudates (for example 20180146632A1 to Meron), or environmental impact on the probe due to lack of sealant or insulation (for example U.S. Pat. No. 8,695,407 to Strook). U.S. Pat. No. 9,374,950 to Upadhyaya teaches a system that determines plant water needs via monitoring leaf temperatures. However, that system suffers from leaf to leaf variance.

Due to the lack of reliably effective plant hydration monitoring options, grower opinions vary on how to measure and manage water, with some growers irrigating by instinctual impulses, and others looking to historical data or soil monitoring systems for insight.

Thus, there is still a need in the art for an improved installation method for a plant hydration monitoring system.

### SUMMARY OF THE INVENTION

The present invention provides methods for installing a plant hydration status sensor into a plant (e.g., woody plants, annuals (corn, soybeans), perennials) to provide reliable plant hydration data. One method comprises (i) creating a hole from an exterior surface of the plant to a depth that exposes the water tissue of the plant (e.g., xylem), (ii) disposing a sleeve within the hole, (iii) disposing a plant hydration status sensor within the sleeve, and (iv) disposing a sealant about the plant hydration status sensor and the sleeve. It should be appreciated that disposing the sleeve and plant hydration status sensor within a hole that extends to (i.e., extends to a point within the plant where the water tissue is exposed) or within water tissue of the plant provides more accurate data from the sensor. It should be further appreciated that disposing a sealant about the plant hydration sensor and the sleeve provides protection to the sensor by forming a barrier between the sensor and outside environment conditions (e.g., plant exudates or other natural compounds, pesticides or other unnatural compounds, and environmental conditions).

It is contemplated that the method can further comprise disposing a slurry within the hole, such that the volume of the hole is occupied entirely by (i) a portion of the sleeve, (ii) a portion of the plant hydration status sensor, and (iii) the slurry. The slurry can contain hydrophilic nanoparticles. It should be appreciated that the slurry provides for a more consistent fluid path between the water tissue and sensor. It should be further appreciated that the inclusion of hydrophilic nanoparticles within the slurry provide for the attaining of a neutral water potential within the sleeve hole, and thus allow for more accurate data obtained by the sensor. In other words, the water potential in the sleeve hole will typically be the water potential of the plant. In exemplary embodiments, the slurry will not affect the water potential between the sleeve hole and plant, and thus will be a non-factor in the measurement of the water potential within the sleeve hole.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a depiction of slurry disposed within a sleeve hole that extends from an exterior surface of the plant to a depth that exposes the water tissue.

FIG. 2 is a depiction of a plant hydration status sensor and a sleeve at least partially disposed within the plant, and a retaining device coupled to the plant hydration status sensor.

FIG. 3 is a depiction of the plant hydration status sensor, at least partially disposed within the plant, covered by insulation, whereby the plant hydration status sensor is shown via a cutaway view.

FIG. 4a is a depiction of a slurry with hydrophilic nanoparticles as a suspension.

FIG. 4b is a depiction of the slurry with hydrophilic nanoparticles of FIG. 4a as a porous matrix.

### DETAILED DESCRIPTION

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps can be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Also, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

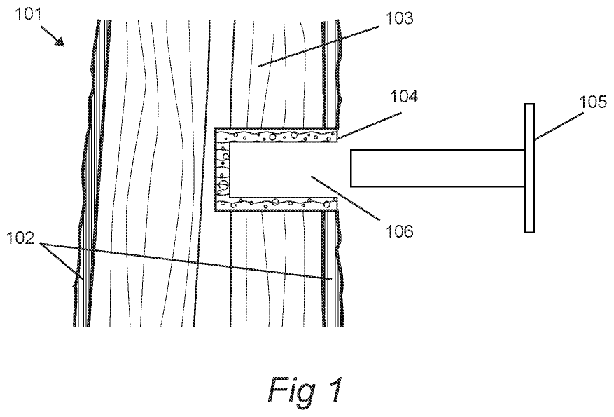

FIG. 1 shows a plant 101 having a bark region 102 and water tissue 103. Hole 106 is produced through bark region 102 to a depth that exposes water tissue 103. As shown in FIG. 1, hole 106 extends within water tissue 103, preferably within water tissue 103 at a depth of at least 1 mm from the beginning of water tissue 103. However, it is contemplated that hole 106 can extend to water tissue 103, but not within water tissue 103 (i.e., extend through all layers external to the water tissue of the plant). For example, hole 106 can extend through layers of a plant to expose a vascular layer that transports water throughout the plant. In another example, hole 106 can extend through the bark, phloem, and cambium layers of a tree, but not the xylem layer, to thereby expose the xylem of a tree. Once hole 106 is created, a slurry 104 is disposed within hole 106.

As shown in FIG. 1, plant 101 could be a tree having the bark, phloem, cambium, and xylem layers. However, it is contemplated that plant 101 could be any type of woody plant, annual (e.g., corn, soybeans), or perennial. Regardless of the plant type, a hole can be created through layers of the plant to a depth within or to water tissue to thereby expose the water tissue for installation of a sleeve, a plant hydration status sensor, and slurry as described herein.

Hole 106 is typically produced via a hole producing device 105, which could be a manually or power driven. For example, hole producing device 105 could be a cork borer or end mill, which are manually driven devices. It is contemplated that a manually driven device can cut through bark region 102 and stop at or within water tissue 103 to produce hole 106. This cutting method of utilizing a manually driven device accurately and consistently provides access to the most active water-carrying tissue (i.e. the outermost xylem), due to the more precise nature of a handheld, manually operating tool. Further, a manually driven device also requires no power source at the installation site, which is advantageous given the lack of easily accessible power in some agricultural settings, such as a vineyard or an orchard.

In exemplary embodiments, positioning of the hole producing device 105 can be facilitated using a bark gauge to measure the thickness of bark region 102, and securing an insertion guide to plant 101. The measurement of the thickness of bark region 102 provides a depth at which the bark region 102 ends and water tissue 103 begins, such that a user can use the measurement of depth to produce hole 106 via hole producing device 105. In certain embodiments, an insertion guide can be secured onto plant 101 via (1) friction upon at least a portion of bark region 102 (e.g., using a clamping device) or (2) insertion into at least a portion of bark region 102 (e.g., using a fastener). An insertion guide should provide alignment of hole producing device 105 with the desired site for hole 106.

Hole 106 could be at least 1 mm in depth measured from the exterior of bark region 102, and in other embodiments, hole 106 can have a depth of at least 15 mm. Hole 106 can be situated such that hole 106 is perpendicularly aligned with plant 101 as shown in FIG. 1. However, it is contemplated that hole 106 can form another angle with plant 101. The depth of hole 106 may vary according to factors, including intrinsic factors such as the taxonomy of plant 101 or characteristics of plant 101 (e.g., size, shape), or extrinsic factors such as weather or nearby human activity.

Hole 106 can have an end surface that is distal to the exterior surface of plant 101 that is flat, curved, or some other topography. In some embodiments, the end of hole 106 is abraded with an abrasion tool to remove inhibiting factors. Suitable abrasion tools include spatulas and knives.

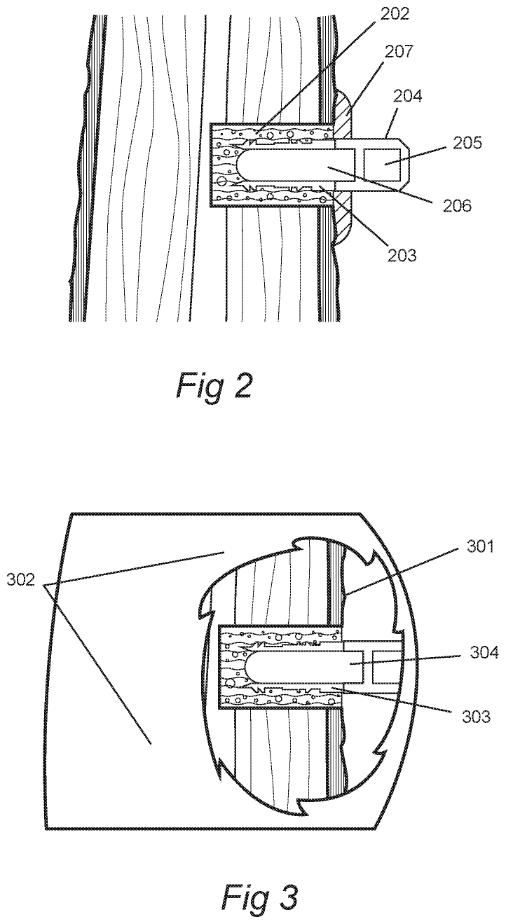

FIG. 2 shows a sleeve 203 disposed within hole 106 that contains slurry 202. It is contemplated that a lubricant can be applied to hole 106 to thereby assist in disposing sleeve 203 within hole 106. As shown in FIG. 2, hole 106 can be filled with an amount of slurry 202 to fill the area between (i) an outer boundary of hole 106, and (ii) sleeve 203 and plant hydration status sensor 206. In other words, sleeve 203, plant hydration status sensor 206, and slurry 202 occupies the entire volume of hole 106. It should be appreciated that providing slurry 202 as shown in FIG. 2 provides a uniform fluid path between the plant hydration status sensor 206 and water tissue 103, which improves data obtained from the sensor. However, in other embodiments, slurry 202 can fill less volume of hole 106.

A retaining device 204 is coupled to sleeve 203 to thereby retain the sleeve 203 within hole 106. It is contemplated that retaining device 204 can advantageously include a biasing component 205, which can be static, such as a nail or screw, or dynamic, such as a spring or elastomer. For example, retaining device 204 can be a threaded cap with a spring as its biasing component 205 such that the spring biases sleeve 203 and plant hydration status sensor 206 towards plant 101 to keep sleeve 203 in close contact plant 101. In preferred embodiments, retaining device 204 allows plant hydration status sensor 206 to follow the expansion or contraction of plant 101.

It should be appreciated that seal 207 can be flexible or dynamic to allow axial movement of plant hydration status sensor 206. Seal 207 can be effected in any suitable manner, including using a highly viscous composition and/or a solid seal. Suitable viscous compositions include greases and/or another lubricants. Suitable solid seals include one or more of parafilm, and an O-ring and a gasket.

Contemplated compositions of seal 207 include an epoxy or other aqueous solvents. For example, contemplated seal compositions can be Loctite® Epoxy Instant Mix™ 1 Minute or J-B Weld® Waterweld™ Epoxy Putty. However, other seal compositions can be used. It should be appreciated that aqueous solvents are disposed about the water tissue 103, and thus do not inhibit accurate measure of water potential within the water tissue 103. In some embodiments, seal 207 is pliable to allow for movement of the plant 101, and is capable of adhering to portions of plant 101 that may be wet so as to prevent intrusion of inhibiting factors into the water tissue 103.

Seal 207 also functions to at least partially exclude inhibiting factors from hole 106 that can damage or alter readings from plant hydration status sensor 206. Contemplated inhibiting factors include plant exudates or other natural compounds, pesticides or other unnatural compounds, and environmental conditions (e.g., rain, snow, insects).

It is contemplated that seal 207 comprises compositions that are water-proof or water-resistant. Further, seal 207 can comprise compositions that can cure in less than completely dry curing conditions. For example, if at least a portion of plant 101, to which the seal composition is being applied, is at least partially wet, the curing process can still proceed. It is contemplated that seal 207 can comprise compositions that can cure in less than five minutes, and in other embodiments, in less than two minutes.

It should be appreciated that seal 207 retains at least some substances within the hole 106 to thereby inhibit changes in the water potential of the hole 106.

FIG. 3 shows insulation 302 used to protect sleeve 303 and plant hydration status sensor 304. Insulation 302 can protect sleeve 303 and plant hydration status sensor 304 from both natural and unnatural external factors, including for example, weather, and animal or human activity.

Insulation 302 preferably comprises some combination of an inner and outer materials that add insulation. Contemplated inner materials include cotton batt, fiberglass or other fibrous materials, as well as calcium silicate or other non-fibrous materials. Contemplated outer materials include aluminum (e.g., a thin film of aluminum), stainless steel or other metallic materials, as well as wood, plastic or other non-metallic materials. It should be appreciated that contemplated outer materials comprise a reflective covering to reflect thermal energy radiated onto the insulation (e.g. sunlight), thus providing the benefit of reducing possibility of erroneous readings from the sensor heating up in response to radiated thermal energy. Additionally, it is contemplated that one or more of the inner and outer materials can be strong enough to shield sensor 304 and sleeve 303 from the outside environment (e.g., impact from a fallen branch).

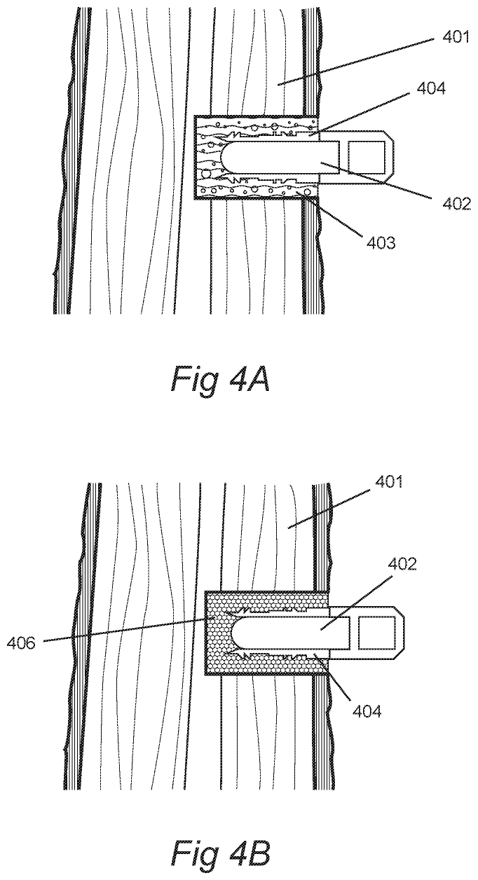

FIG. 4a depicts sleeve 404 and plant hydration status sensor 402 disposed within hole 106, and slurry 403 depicted as a suspension of nanoparticles. Here, slurry 403 provides a fluid path between plant hydration status sensor 402 and water tissue 401. It is contemplated that water tissue 401 can exhibit a negative atmospheric pressure. For example, water tissue 401 of the tree can exhibit a negative atmospheric pressure of approximately 0 to 50 atmospheres. In another example, water tissue 401 of the tree can exhibit a negative atmospheric pressure of approximately 0 to 100 atmospheres (e.g., plants in the desert can exhibit this negative atmospheric pressure range). It should be appreciated that the negative atmospheric pressure can remove fluid from the slurry 403 such that the nanoparticles of slurry shown in FIG. 4a form porous matrix 406 shown in FIG. 4b.

As shown in FIG. 4b, sleeve 404 and plant hydration status sensor 402 are disposed within hole 106, and slurry 403 has a porous matrix 406. In exemplary embodiments, at least some of the pores of the porous matrix 406 will vary in size. At least some variance in pore size is advantageous because it facilitates retention of different amounts of fluid at different pressures before the fluid empties from the pores. For example, a 100 nm pore retains water at −25 atmospheres of pressure, and empties at −30 atmospheres of pressure. As another example, a 50 nm pore retains water at −50 atmospheres of pressure.

Slurries having a majority of 100 nm pores are preferred for plants that experience less water stress (i.e., higher pressures within the plant). For example, slurries having a majority of 100 nm pores can be used for trees native to rainforests and other plants in moisture-rich environments that typically exhibit less water stress (e.g., −3 atmospheres). Slurries having a majority of 50 nm pores are preferred for plants that experience more water stress (i.e., lower pressures within the plant). For example, slurries having a majority of 50 nm pores can be used for cacti and other plants in moisture-poor environments (e.g., −60 atmospheres).

The nanoparticles in slurry 403 are preferably hydrophilic. Hydrophilic nanoparticles can be metallic or non-metallic. Contemplated metallic nanoparticles include aluminum oxide, and suitable non-metallic nanoparticles include silicone.

Thus, specific compositions and methods of plant hydration monitoring have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A method for installing a plant hydration status sensor into a plant having water tissue, comprising:
creating a hole from an exterior surface of the plant to a depth that exposes the water tissue;
disposing a sleeve within the hole;
disposing a plant hydration status sensor within the sleeve; and
disposing a sealant about the plant hydration status sensor and the sleeve.

2. The method of claim 1, wherein the depth is to an outer boundary of the water tissue.

3. The method of claim 2, wherein the water tissue is a xylem of the plant.

4. The method of claim 1, wherein the depth is at least 1 mm into the water tissue.

5. The method of claim 4, wherein the water tissue is a xylem tissue of the plant.

6. The method of claim 1, further comprising disposing a slurry containing hydrophilic nanoparticles within the hole, such that at least some of the slurry is disposed between (i) the water tissue and (ii) one or more of the sleeve and the plant hydration status sensor.

7. The method of claim 6, wherein the hole defines a volume, and wherein the volume is occupied entirely by (i) a portion of the sleeve, (ii) a portion of the plant hydration status sensor, and (iii) the slurry.

8. The method of claim 6, wherein at least some of the hydrophilic nanoparticles comprise alumina.

9. The method of claim 6, wherein at least some of the hydrophilic nanoparticles are configured to form a matrix having a plurality of pores when the water tissue pulls fluid from the slurry.

10. The method of claim 9, wherein the plurality of pores has an average size of less than 50 nm.

11. The method of claim 1, further comprising applying a lubricant into the hole to thereby assist in disposing the sleeve within the hole.

12. The method of claim 1, further comprising (i) securing an insertion guide onto the plant to provide alignment for a hole producing device to create the hole, and (ii) inserting the hole generating device through the insertion guide to create the hole.

13. The method of claim 1, wherein the sealant comprises an O-ring.

14. The method of claim 1, wherein the sealant comprises a chemical sealant.

15. The method of claim 1, further comprising coupling the sleeve to a retaining device configured to bias the plant hydration status sensor to remain within the sleeve.

16. The method of claim 15, wherein the retaining device comprises an elastic member to bias the plant hydration status sensor to remain within the sleeve.

17. The method of claim 1, further comprising installing insulation to cover one or more of the sleeve, the plant hydration status sensor, and the sealant.

18. The method of claim 17, wherein the insulation comprises a plurality of cellulose fibers.

19. The method of claim 17, wherein the insulation comprises a reflective covering.

20. The method of claim 1, wherein the plant comprises bark, phloem, and cambium layers, and wherein the hole is created to penetrate through the bark, phloem, and cambium layers to thereby expose the water tissue.
